(19) United States
(12) Patent Application Publication
(10) Pub. No.: US 2004/0157953 A1
(43) Pub. Date: Aug. 12, 2004

(54) Materials for filling cavities in the body

(76) Inventor: Christopher H. Porter, Woodenville, WA (US)

Correspondence Address: Gerald F. Swiss, Foley & Lardner LLP, Three Palo Alto Square, 3000 El Camino Real, Suite 100, Palo Alto, CA, 94306-2121, US

(21) Appl. No.: 10/687,518

(22) Filed: Oct. 15, 2003

Related U.S. Application Data
(60) Provisional application No. 60/418,251, filed on Oct. 15, 2002.

(51) Int. Cl. A61F 2/00

(52) U.S. Cl. 523/115

(57) ABSTRACT

Materials for filling cavities in the body are disclosed which have very high viscosity when in the cavity and a lower viscosity when being delivered to the cavity.

### CROSS-REFERENCE TO RELATED APPLICATIONS

This application claims the benefit under 35 U.S.C. § 119(e) of U.S. Provisional Application 60/418,251, filed Oct. 15, 2002,

### BACKGROUND OF THE INVENTION

When using fillers in cavities in the body, especially brain aneurysms, it is highly desirable that the filling material has a high viscosity to ensure that the material, after delivery, will not flow out of the cavity. It can be stated as a general rule, the higher the viscosity of the fluid in the aneurysm, the better.

This desirability of this high viscosity is offset by the problem of delivering these materials. They are necessarily transferred to distant locations through long tubes with very small diameter. These tubes need to be passed through torturous vessels to access the aneurysm. The transport of high viscosity materials through these tubes results in high shear stresses. This results in very high delivery pressures and requires very robust catheters. Robust catheters, by necessity, have thick wall and accordingly are not very flexible. These types of catheters are hard if not impossible to be passed through the tortuous vasculature upstream of the aneurysm. As a general rule, the lower the viscosity of the fluid being delivered, the better.

In the systems used today, there is a trade-off between the viscosity of the material in the aneurysm (the higher the better) and the viscosity of the delivery material (the lower the better). Generally this trade-off is resolved by using a material that has some compromise viscosity. Even at this compromise viscosity, one has material that behaves marginally in the aneurysm which also requires expensive and stiff delivery catheters.

### DETAILED DESCRIPTION OF THE INVENTION

Pseudo-Plastic Materials

General

Pseudo-plastic materials are ones whose viscosities decrease with shear. The apparent viscosity of these materials decreases when they are pushed through tubes. The apparent viscosity increases once the flow has stopped. Some of these changes are time dependent (thixotropic) and some are almost instantaneous. Both types of materials can be used.

The characteristics of these types of fluid are shown if FIG. 1. These fluids have a low viscosity when being delivered (some shear rate) and a higher viscosity after they have exited the catheter (no shear rate).

The advantage of using pseudo-plastic solutions is obvious from the above figure. Generally, the allowable viscosity of the fluid is determined by what can be delivered in a practical system (catheter with a given length and diameter) at a practical flow rate. This will define a certain shear rate (Point B).

The viscosity of the fluid in the aneurysms is the viscosity of that material at zero-shear rate. When using a pseudo-plastic material, the viscosity at rest, Point A, is much greater than the viscosity of a Newtonian material, point C. Use of a pseudo-plastic instead of a Newtonian fluid will allow one with a given delivery system to deliver a material with a higher at-rest viscosity. The higher the degree of shear thinning the better.

Bingham Plastics

Bingham plastics are materials that do not flow at all until you they experience a certain critical stress. Once this critical stress has been reached, they flow freely. They can be considered to be a special case of pseudo-plastic materials.

Toothpaste is an example of a Bingham plastic. When it is not squeezed (stressed) it stays put and acts like it has an infinite viscosity. Once you get it flowing, if flows quite freely. Once it gets where you want it to go, the toothbrush, it then acts like it has an infinite viscosity again. A Bingham plastic could be an ideal material for filling aneurysms.

Sources of Materials

Methods for obtaining solutions that exhibit pseudo-plastic behavior include:

1. Formulating a compound which exhibits the behavior and using it directly

2. Adding substances to an existing non/less pseudo-plastic solution (so call thickening agent).

Examples of these pseudo-plastic enhancing agents include:

1. Adding small fillers to the material

i. Calcium carbonate

ii. Barium Sulfate

iii. Ground up filler agent itself

iv. Carbon beads

v. Silica

1. fumed

2. small particles

vi. TiO2

vii. Magnetic materials

viii. Etc.

2. Adding a quantity of dispersed fiber

3. Adding highly pseudo-plastic polymer solutions.

4. Combinations of the above.

Transitional Systems (Switched Systems)

There are other ways to cause a low viscosity liquid to transition to a high-viscosity material when delivered. These include materials that:

1. change an intrinsic property (gets more viscous due to a reduction of temperature)

2. change state (for example: from a liquid to solid),

3. undergoes a phase transition (visco-elastic material to a glass)

4. change in structure (the materials increased its molecular weight or cross links).

Several means of initiating these transitions are outlined below.

1 Delivering the material at one temperature (generally warm) and cooling it in the cavity.

2 Initiate a chemical reaction—i.e. put an initiator in a pre-polymer, and then imitating a chemical reaction as it enters the aneurysm.

a This can be done continuously as it enters

b or alternately delivering it and then initiating the reaction.

Reactable Materials

Generally, the reactable materials would be pre-polymers or monomers with an initiator in them that is activated when the material enters the aneurysm. There are numerous ways of initiating a reaction of the mass exiting the catheter including:

1 Heat

2 Light (see FIG. 2)

3 Addition of a second compound

4 Loss of a material by diffusion

5 Magnetic energy

6 Time (use a material that sets up with a known initiation time)

7 Etc.

Ideally the reactable material is a substance that if it does escape the cavity and goes into the blood stream, it is relative non-toxic and it dissolves or rapidly breaks down as not to form emboli. Example of these includes blood soluble pre-polymers that cross link when reacted into a non-soluble form. Another way of doing this is to use soluble material to fill the aneurysm and then capping it with a non-soluble material.

### SUMMARY OF THE INVENTION

The ideal solution to the problem is to use a material that has a very high viscosity when it is in the aneurysm and that has a low viscosity when it is being delivered.

There are several ways one could develop a material that has a low viscosity when being delivery and a high viscosity at the delivery point. These include:

1. using a highly pseudo-plastic material or a Bingham plastic as the filler.

2. using a low-viscosity material and then rapidly switching this to a high-viscosity material in the aneurysm.

Additional advantages and novel features of the invention will be set forth in part in the description which follows, and in part will become apparent to those skilled in the art upon examination of the following, or may be learned by practice of the invention.

### BRIEF DESCRIPTION OF THE DRAWINGS

FIG. 1. The viscosity of the pseudo-plastic solution gets less at the shear rate (flow through a tube) increases. The viscosity of a Newtonian fluid stays constant. A pseudo-plastic solution has a higher viscosity at rest than it does when it flows.

FIG. 2. A schematic view of a composition of the invention being used with light activation to provide a high viscosity reacted mass.

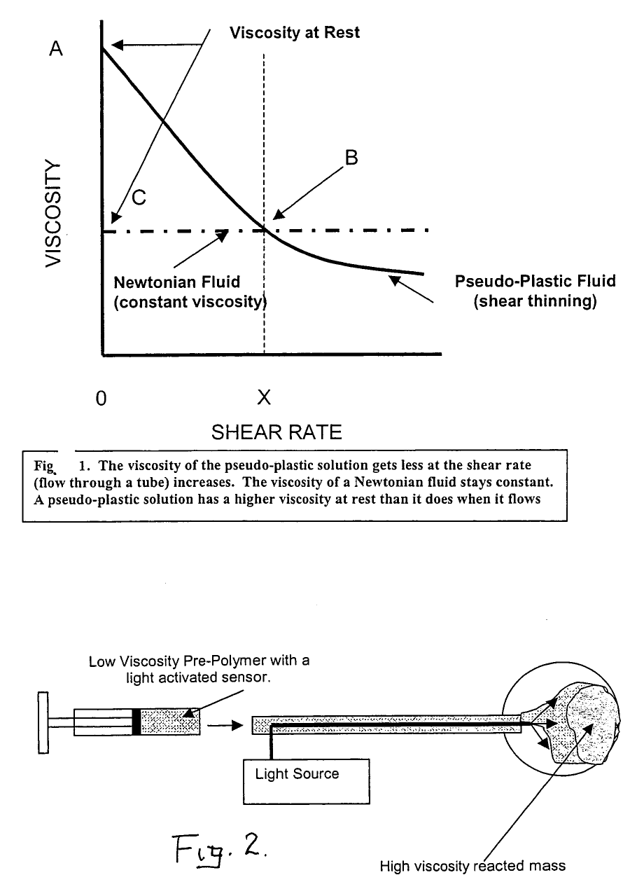

I claim:

1. A material for filling a cavity in the body which has a very high viscosity when in the cavity and a lower viscosity when being delivered.

2. The material of claim 1 comprising a highly pseudo-plastic filler.

3. The material of claim 1 comprising a Bingham plastic filler.

4. A method of filling a cavity in a body comprising delivering a material of claim 1, 2 or 3 to the cavity at a lower viscosity and retaining the material in the cavity at a high viscosity.
